(19) United States
(12) Patent Application Publication
(10) Pub. No.: US 2025/0392234 A1
(43) Pub. Date: Dec. 25, 2025

(54) BATTERY-FREE, SELF-POWERED SENSOR DEVICE AND MONITORING SYSTEM

(71) Applicants: Noora ALMARRI, London (GB); Fawaz NAHAB, London (GB)

(72) Inventors: Noora ALMARRI, London (GB); Fawaz NAHAB, London (GB)

(21) Appl. No.: 19/245,772

(22) Filed: Jun. 23, 2025

Related U.S. Application Data
(60) Provisional application No. 63/662,468, filed on Jun. 21, 2024.

(51) Int. Cl. H02N 2/18; A61B 5/00; A61B 5/02

(52) U.S. Cl. CPC H02N 2/186; A61B 5/0004; A61B 5/02028; A61B 5/6869; A61B 5/7225; A61B 2560/0214; A61B 2560/0252; A61B 2562/028

(57) ABSTRACT

A self-powered sensor device and monitoring system. The self-powered sensor device includes a harvester module having a power harvesting element that is arranged and disposed to harvest power from induced mechanical strain, a sensor module having a sensor element and a controller module having circuitry operably connected to the harvester module and the sensor module. The controller module is configured to receive power from the power harvesting element, to receive sensor signals from the sensor element, and to wirelessly communicate an output signal corresponding to the sensor signals. The controller module is powered continuously with power harvested from the power harvesting element to generate and communicate the output signal. The self-powered sensor device is suitable for use as an implantable cardiac sensor.

### RELATED APPLICATIONS

This application claims priority to U.S. Provisional Patent Application 63/662,468, filed Jun. 21, 2024, entitled, “HEARTBEAT-POWERED IMPLANTABLE INTEGRATED SYSTEM FOR SENSING CARDIAC OUTPUT AND HEART STIFFNESS”, which is incorporated by reference herein in its entirety.

### FIELD OF THE INVENTION

The present disclosure is generally directed to a self-powered sensor device integrating energy harvesting, sensing, and real-time data telemetry.

### BACKGROUND OF THE INVENTION

Implantable sensors hold significant promise for transforming healthcare by enabling continuous monitoring, precision diagnostics, and timely therapeutic interventions. However, a fundamental challenge facing implantable technologies is the reliable and safe delivery of power.

Traditionally, these devices have relied on batteries to support continuous or periodic operation. While batteries offer a straightforward and self-contained power solution, they come with several significant drawbacks. First, batteries have a finite operational lifespan and require periodic recharging or eventual replacement-often through invasive surgical procedures. Second, their size and chemical composition can pose safety hazards; fractured or degraded batteries may leak harmful substances or fail unpredictably. These concerns are particularly problematic for long-term or deeply implanted systems, where battery replacement or repair is not trivial. Moreover, the need to recharge batteries frequently places an additional burden on patients and caregivers, and the energy storage capacity inherently limits device miniaturization. Likewise, the size and space required to include a battery further limits device miniaturization.

To address some of these limitations, certain wirelessly powered implants have been developed. By removing the internal battery, such systems eliminate the associated safety and longevity issues. However, these devices depend on external power transmitters to function, which introduces new complications. External devices can be cumbersome, easily damaged, or cosmetically unappealing, and they require the patient to be within range of the transmitter to operate the implant. For applications that demand continuous monitoring or real-time alerts, such periodic connectivity is inadequate and potentially dangerous.

Energy harvesting from the body or ambient environment is a desirable source of power. Currently, the human body offers few reliable ambient energy sources. For example, deep within tissue, light is largely unavailable; temperature gradients are minimal and inconsistent; and body motion or vibration is unpredictable and varies with activity level, health status, and sleep and yields low power output. Despite these limitations, researchers have explored various biomechanical energy harvesting strategies, but found unacceptable performance over time. Other attempts at generating power internally in the body have required long implanted leads to transmit power elsewhere in the body, complicating both the surgical procedure and the system's reliability.

Piezoelectric harvester devices have simple configurations and potentially a high conversion efficiency compared to other potential in-body energy harvesting approaches. However, current systems lack the multidisciplinary solutions required to combine electrical and mechanical analyses to optimize power harvesting and sensing methods within the body. Prior studies have investigated piezoelectric energy harvesting using brittle lead zirconate titanate (PZT) ceramics to capture mechanical energy from cardiac motion. However, the use of PZT as a material may present biocompatibility and mechanical durability challenges for chronic implantable use and would require encapsulation strategies that limit overall power harvesting efficiency.

Cardiovascular diseases are the leading cause of premature death worldwide. Existing implantable cardiovascular devices are limited by reliance on battery-powered operation, necessitating periodic surgical replacement and reducing long-term system viability. Furthermore, current systems often lack the capability for continuous, real- time biomechanical assessment of cardiac function, particularly for stiffness and output metrics.

Cardiovascular diseases such as coronary artery disease, heart failure, stroke and peripheral arterial disease are the leading causes of premature death and disability, and the rise of medical costs worldwide. Heart failure alone affects more than 65 million people worldwide requiring patients to undergo heart transplantation and leading to 1 out of 4 people prone to develop heart failure. Cardiovascular electronic devices (CEDs) such as implantable pacemakers, implantable cardioverter-defibrillators (ICDs), cardiac resynchronization therapy have been hugely beneficial to cardiovascular health and has reduced morbidity and mortality associated with cardiovascular disease, however, accuracy in disease detection remains a challenge. Another challenge is the battery replacement surgeries.

Cardiovascular allograft vasculopathy (CAV) is a condition that affects transplanted hearts, causing the blood vessels that supply the heart muscle to narrow and eventually block. It is unclear what causes CAV but there are potential leading causes including viral infections and the use of immunosuppressive drugs. In an advanced CAV, retransplantation is the only viable solution; however, early diagnosis of heart stiffness could reduce the need for re-transplantation through personalized management and prevention strategies, allowing better clinical outcome. Better understanding of potential treatments could be achieved by an intracardiac pressure sensor in a transplanted heart for an extended period.

While batteries have traditionally powered implantable devices, their limited life, safety risks, and maintenance demands make them increasingly unsuitable-particularly as implants are expected to operate autonomously and over long durations. Alternatives such as wireless power transfer and energy harvesting show potential but face significant challenges related to usability, consistency, and anatomical limitations. Accordingly, it is desirable in the art to provide a sensor device and monitoring system having a reliable power source that overcomes hurdles and ensures safety, reliability, and patient comfort across a wide range of clinical scenarios. It is desirable to increase the longevity of an implant by providing continuous power, particularly where battery replacement is unfeasible.

What is needed is a self-powered sensor device and monitoring system capable of autonomously harvesting and managing sub-microwatt levels of energy generated from, for example, cardiac motion to fully operate sensing, signal processing, and wireless telemetry modules, overcoming prior limitations in power sufficiency, safety, and device longevity to provide untethered operation that does not suffer from the drawbacks of the prior art. Other features and advantages will be made apparent from the present specification. The teachings disclosed extend to those embodiments that fall within the scope of the claims, regardless of whether they accomplish one or more of the aforementioned needs.

### SUMMARY OF THE INVENTION

The present invention provides a sensor device and monitoring system that harvests mechanical strain from a target substrate to generate power capable of operating continuously at nanowatt power levels, with real-time wireless telemetry, and applicable across various domains. The sensor device and monitoring system provides desirable wireless sensing of conditions in applications medical implants to industrial monitoring. The sensor device according to the present disclosure includes a power harvesting element (harvester) that continuously powers control circuitry (circuitry), which in turn conditions the sensor signals (via sensor element) and transmits data wirelessly.

An embodiment according to the present disclosure includes a self-powered sensor device. The self-powered sensor device includes a harvester module having a power harvesting element that is arranged and disposed to harvest power from induced mechanical strain, a sensor module having a sensor element and a controller module having circuitry operably connected to the harvester module and the sensor module. The controller module is configured to receive power from the power harvesting element, to receive sensor signals from the sensor element, and to wirelessly communicate an output signal corresponding to the sensor signals. The controller module is powered continuously with power harvested from the power harvesting element to generate and communicate the output signal.

Another embodiment according to the present disclosure includes a monitoring system. The monitoring system includes a target substrate, a self-powered sensor device mounted on a flexible carrier. The sensor device and flexible carrier are positioned on the target substrate. The self-powered sensor device includes a harvester module having a power harvesting element that is arranged and disposed to harvest power from induced mechanical strain, a sensor module having a sensor element and a controller module having circuitry operably connected to the harvester module and the sensor module. The controller module is configured to receive power from the power harvesting element, to receive sensor signals from the sensor element, and to wirelessly communicate an output signal corresponding to the sensor signals. A signal receiver is configured to receive the output signal from the sensor device. The controller module is powered continuously with power harvested from the power harvesting element to generate and communicate the output signal.

Another embodiment according to the present disclosure includes a biocompatible, flexible implantable sensor device configured to harvest mechanical energy from cardiac motion. The harvested energy, captured at sub-microwatt levels, is autonomously managed to operate integrated sensing, signal acquisition, and wireless telemetry modules without reliance on external power sources. The system enables high-accuracy real-time monitoring of myocardial stiffness and cardiac output, addressing previous limitations in continuous implantable monitoring due to power insufficiency.

Another embodiment according to the present disclosure is an implantable cardiac sensor. The implantable cardiac sensor includes a biocompatible flexible carrier configured for implantation on a myocardial surface, a harvesting module having a power harvesting element, a sensor module having a sensor element and a controller module having circuitry operably connected to the harvester module and the sensor module. The harvesting module includes one or more piezoelectric elements selected from poly(vinylidene fluoride) barium titanate (PVDF-BaTiO₃) fibers, poly(vinylidene fluoride) (PVDF) fibers, poly(vinylidene fluoride) (PVDF) films, polymethyl methacrylate (PMMA), lead zirconate titanate (PZT) films, and combinations thereof. The sensor element includes piezoelectric MEMS sensors configured to detect myocardial deformation, filtering out heart signals. The controller module is configured to receive power from the power harvesting element, to receive sensor signals from the sensor element, and to wirelessly communicate an output signal corresponding to the sensor signal. The controller module is powered continuously with power harvested from the power harvesting element to generate and communicate the output signal. The circuitry includes maximum power point tracking (MPPT) control circuitry and power conversion circuitry configured to regulate harvested electrical energy for continuous battery-free system operation at ultra-low power levels and derive ejection fraction, cardiac stiffness and cardiac output parameters to communicate as data in the output signal.

The system comprises distinct piezoelectric components configured for separate energy harvesting and biomechanical sensing functions. A piezoelectric energy harvesting module captures mechanical energy from myocardial motion to generate electrical power for device operation. Independently, a piezoelectric MEMS-based sensor array monitors myocardial deformation to extract cardiac stiffness and output parameters. Both subsystems utilize piezoelectric transduction mechanisms, allowing fully self-powered operation without external power sources.

Embodiments of the present invention enable real-time monitoring of cardiac stiffness and output in patients with cardiovascular diseases, addressing prior limitations wherein implantable systems lacked sufficient power capacity for continuous biomechanical sensing and wireless telemetry without external power sources or frequent battery replacement surgeries.

Other features and advantages of the present invention will be apparent from the following more detailed description of the preferred embodiment, taken in conjunction with the accompanying drawings which illustrate, by way of example, the principles of the invention.

### BRIEF DESCRIPTION OF DRAWINGS

FIG. 1 schematically illustrates a sensor device according to an embodiment of the present disclosure.

FIG. 2 schematically illustrates a monitoring system according to an embodiment of the present disclosure.

FIG. 3 schematically illustrates an enlarged view of a sensor element and a sensor module of a sensor device according to an embodiment of the present disclosure.

FIG. 4 schematically illustrates an enlarged view of a harvester module and a power harvesting element of a sensor device according to an embodiment of the present disclosure.

FIG. 5 schematically illustrates an enlarged view of controller module according to an embodiment of the present disclosure.

FIG. 6 schematically illustrates an enlarged view of controller module including an exemplary arrangement of circuitry according to an embodiment of the present disclosure.

FIG. 7 schematically illustrates an enlarged view of controller module including another exemplary arrangement of circuitry according to an embodiment of the present disclosure, including an output signal and signal receiver.

FIG. 8 schematically illustrates an enlarged view of controller module including still another exemplary arrangement of circuitry according to an embodiment of the present disclosure.

FIG. 9 schematically illustrates a sensor device having an enlarged view of a sensor element and an enlarged power harvesting element according to another embodiment of the present disclosure.

FIG. 10 schematically illustrates a monitoring system including a receiver patch according to another embodiment of the present disclosure.

FIG. 11 schematically illustrates the signal and data flow within the monitoring system of FIG. 10.

FIG. 12 is a flow chart illustrating a method for fabricating a sensor element according to an embodiment of the present disclosure.

FIG. 13 is a flow chart illustrating a method for fabricating a power harvesting element according to an embodiment of the present disclosure.

FIG. 14 shows the ex-vivo measurement setup with a sensor device 100 according to the present disclosure.

FIG. 15 is a graph showing measured and modelled power output for a 1 MΩ resistive load over a range of heartbeat frequencies from the measurement setup in FIG. 14.

FIG. 16 shows the measured output voltage waveform at 1.02 Hz produced by the harvester module and the rectified voltage, V₀ from the measurement setup in FIG. 14.

FIG. 17 is a graph showing a measured voltage response from the signal acquisition submodule 501 of controller module 103 from the measurement setup in FIG. 14.

FIG. 18 shows an exemplary configuration of a sensor device according to the present disclosure.

FIG. 19 is a graph showing loaded and unloaded voltage measurements from the sensor device shown in FIG. 18.

FIG. 20 shows another exemplary configuration of a sensor device according to the present disclosure.

FIG. 21 is a graph showing loaded and unloaded voltage measurements from the sensor device shown in FIG. 20.

FIG. 22 shows the in-vivo measurement setup with a sensor device 100 according to the present disclosure.

FIG. 23 is a graph showing time-domain signal output from the implanted sensor device 100 in the measurement setup in FIG. 22.

FIG. 24 is a graph showing frequency-domain spectrum of the received signal from the implanted sensor device 100 in the measurement setup in FIG. 22.

FIG. 25 is a graph showing signal amplitude comparison from the implanted sensor device 100 in the measurement setup in FIG. 22.

FIG. 26 is a graph showing peak analysis and tracking over time to detect cardiac anomalies from the implanted sensor device 100 in the measurement setup in FIG. 22.

Wherever possible, the same reference numbers will be used throughout the drawings to represent the same parts.

### DETAILED DESCRIPTION OF THE INVENTION

Embodiments of the present disclosure includes an ultra-low power, modular, self-powered sensor system capable of providing real-time wireless output signals utilizing harvested power. For example, the sensor devices according to the present disclosure may operate continuously at, for example, less than 100 nW, enabling persistent monitoring and data transmission. Embodiments of the present disclosure include real-time signal conditioning and data transmission directly from harvested energy without reliance on external energy storage or intermediate recharge. The sensor device and monitoring system according to the present disclosure may be configured for different sensing modalities (e.g., conditions, such as, pressure, vibration, flow, stiffness), and supports flexible deployment with bluetooth low energy (BLE), ultra-wideband (UWB), or long range (LoRa) transmission protocols. The sensor device and monitoring system according to the present disclosure has the ability to detect, for example, tissue stiffness and cardiac flow changes in real-time—with in-vivo validation, providing early diagnostic insight, particularly for heart failure and other flow-sensitive conditions, which existing systems do not address in a self-powered format. The integrated approach of nano-power+real-time wireless+clinically validated physiological measurements utilized in the sensor device according to the present disclosure provides significant advantages over known sensor systems. This level of integrated sensing, signal processing, and wireless telemetry at sub-100 nW operational levels represents a substantial advance over prior implantable systems, which lacked sufficient energy efficiency to sustain continuous autonomous operation.

Advantages of the sensor system according to the present disclosure include:
Biocompatible and flexible design.
Self-powered by heartbeats, eliminating the need for batteries or other similar energy storage devices.
High accuracy in real-time monitoring of heart parameters.
Integrated system-on-chip for efficient energy harvesting and data transmission.
Potential to improve early detection and management of cardiovascular diseases.
Allows accurate sensing and operation without external calibration.

The sensor device 100 and monitoring system 200 according to the present disclosure are the first heartbeat-powered systems having these advantages. Known systems fail to have the combination of being biocompatible, flexible, lightweight, and battery-free, while being self-powered, easily attachable, and ready to use (doesn't need building or setup to work). With an ultra-Low power requirement. For example, the functionality of the device is distinct from that of existing leadless cardiac pacemakers, such as the Aveir pacemaker single- or dual-chamber systems, by being specifically adapted for mechanical, not electrical, cardiac tissue characterization.

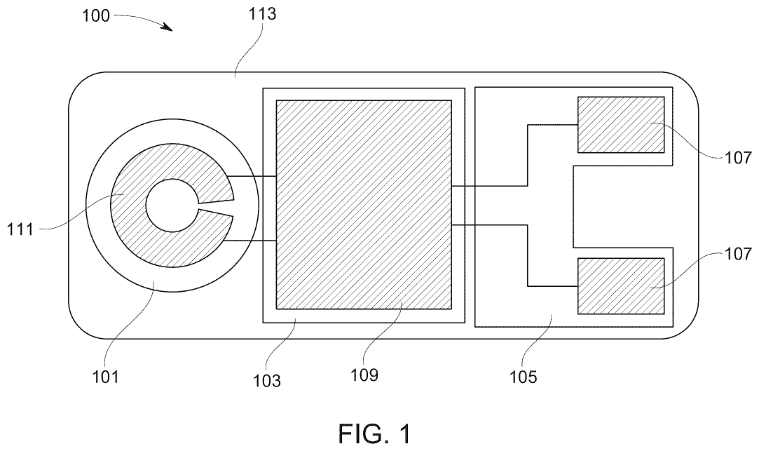

FIG. 1 shows a self-powered sensor device 100 according to an embodiment of the present disclosure. The sensor system 100 includes a harvester module 101, a controller module 103 and a sensor module 105. The harvester module 101 includes a power harvesting element 111. The power harvesting element is arranged and disposed to harvest power from induced mechanical strain.

“Power”, as utilized herein, refers to electrical power (measured in Watts), derived from voltage and current generated via mechanical strain transduction. “Ultra-low power”, as used herein, includes power levels below about 100 nW, with some embodiments operating at <10 nW per circuit block. As utilized herein, “harvesting power”, is defined as including the converting of an induced mechanical strain from a mechanical stress, such as squeezing, bending, pressing or otherwise moving a power harvesting material or from friction into an electrical voltage. Triboelectric power harvesting may result from induced mechanical strain also includes power resulting from triboelectric effects, such as relative movement between surfaces in frictional contact. Triboelectric power harvesting is especially effective during contact-separation or sliding motion.

In certain embodiments, the configuration of the power harvesting element 111 utilizes both triboelectric and piezoelectric effects of the material of the power harvesting element 111. The triboelectric effect, for example, can produce large voltage peaks, which allows for miniaturization of the device. An interdigital electrode design may be utilized to capture triboelectricity by allowing a center electrode to float from the material. The power harvesting element 111 may include, for example, a piezoelectric material. The power harvesting element 111 may include one or more piezoelectric materials selected from poly(vinylidene fluoride) barium titanate (PVDF-BaTiO₃) fibers, poly(vinylidene fluoride) (PVDF) fibers, poly(vinylidene fluoride) (PVDF) films, polymethyl methacrylate (PMMA), lead zirconate titanate (PZT) films, and combinations thereof. A particularly suitable piezoelectric material may include, for example, a piezoelectric fibrous membrane including PVDF-BaTiO3. The poly(vinylidene fluoride) barium titanate (PVDF-BaTiO3) nanofiber composites have a high degree of flexibility and biocompatibility. In addition, in certain embodiments, the power harvesting element 111 is devoid of lead zirconate titanate (PZT). In other embodiments, the sensor device 100 is implantable in the human body, for example, via keyhole surgery. For example, the sensor device 100 may be configured for temporary implantation in a donor heart's left ventricle during ex vivo perfusion or preservation for transplantation viability assessment.

In certain embodiments, the sensor device 100 is lightweight, having a total mass of less than about 0.4 grams. In various alternative embodiments, the total mass may be less than about 0.3 grams, less than about 0.2 grams, or even less than about 0.1 grams, depending on the configuration and intended application.

The sensor module 105 includes one or more sensor elements 107. The sensor elements 107 may include any device, material, or component capable of generating a signal in response to a physiological or mechanical condition. For example, in one embodiment, the sensor element 107 includes one or more piezoelectric microelectromechanical system (MEMS) sensors. In other embodiments, the sensor element 107 may include capacitive sensors, resistive strain gauges, optical sensors (e.g., fiber Bragg gratings), accelerometers, thermistors, or electrochemical sensors, depending on the sensing requirement. Conditions measurable by sensor element 107 may include, for example, pressure, temperature, vibration, flow, stiffness, strain, displacement, acceleration, temperature, Cardiac wall motion velocity, or biochemical markers (e.g., pH, oxygen saturation, lactate levels). These measurable conditions may be used to determine or calculate important physiological parameters, such as muscle stiffness, cardiac tissue deformation, intracardiac pressure, or blood flow velocity. In one embodiment, the sensor element 107 includes at least one MEMS sensor configured to detect mechanical deformation of ventricular tissue during the cardiac cycle.

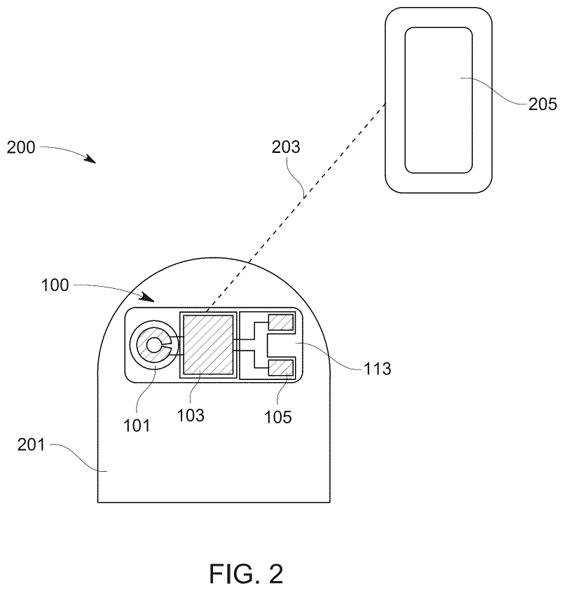

The controller module 103 includes circuitry 109 operably connected to the harvester module 101 and the sensor module 105. The controller module 103 is arranged and disposed and includes circuitry 109 that is configured to receive power from the power harvesting element 111, to receive sensor signals from the sensor element 107, and to wirelessly communicate an output signal 203 (see, for example, FIG. 2) corresponding to the sensor signal. The controller module 103 is powered continuously with power harvested from the power harvesting element 111 to generate and communicate the output signal 203.

As shown in FIG. 2, the sensor device 100 may be utilized in a monitoring system 200 for monitoring a condition on or near a target substrate 201. The monitoring system 200 includes a sensor device 100 mounted on a flexible carrier 113, which is positioned on the target substrate 201. Flexible carrier 113 may be any suitable material providing biocompatibility and thermal stability, such as, but not limited to, polyimide or silicone material. Other flexible carriers may include thin film PCB substrates. These PCB substrates may have a mechanical modulus of up to or about 7 kPa. The target substrate 201 may be any suitable substrate that exhibits vibration, oscillatory motion or other movement capable of inducing strain and has a condition for measurement at or near the target substrate 201. For example, the target substrate 201 may be a heart or other human organ. For example, the target substrate may be the myocardial surface of a heart and the condition may be muscle stiffness. In other embodiments, the target substrate may be a non-medical component, such as a pipe, engine component, construction element or other component that exhibits vibration or oscillatory motion and has a condition that can be measured by sensor device 100. Other applications for use of the sensor device 100 according to the present disclosure may include, for example, vibration-sensitive monitoring in turbines, aircraft wings (e.g., fluttering monitoring), civil infrastructure (e.g., bridges), wearable robotics (e.g., joint motion sensing), biomechanical prosthetics, wind turbines (e.g., rotational vibration sensing) or other applications where battery-free sensing is advantageous.

As shown and described in FIG. 2, the sensor device 100 includes a harvester module 101 having a power harvesting element 111, a sensor module 105 having a sensor element 107, and a controller module 103 having circuitry 109 operably connected to the harvester module 101 and the sensor module 105 and being configured to receive power from the power harvesting element 111, to receive sensor signals from the sensor element 107. The controller module 103 further includes circuitry 109 to wirelessly communicate an output signal 203 corresponding to the sensor signal. The controller module 103, including circuitry 109 is powered continuously with power harvested from the power harvesting element 111 to generate and communicate the output signal 203. The output signal 203 is received by signal receiver 205. Signal receiver 205 may be any device capable of receiving the output signal 203 and analyze, review and/or display data correlated to output signal 203. For example, signal receiver 205 may include a computer, a mobile device, a display device or any other device suitable for analyzing, reviewing and/or displaying data.

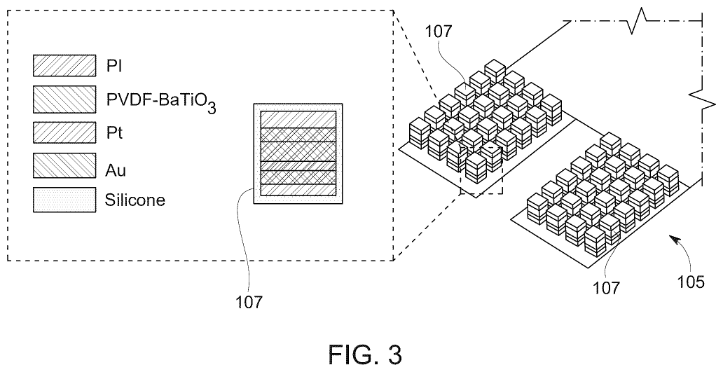

FIG. 3 schematically illustrates an enlarged view of a sensor element 107 and a sensor module 105 of a sensor device 100 according to an embodiment of the present disclosure. In the embodiment shown in FIG. 3, the sensor element includes a layered system including a layer or gold (Au), a layer of platinum (Pt), a layer of PVDF-BaTiO3 and a polyimide layer. The layered system is encapsulated or coated with silicone. The sensor element 107 is not limited to the arrangement shown in FIG. 3. Other suitable sensor elements 107 may include, for example, one or more of a strain gauge, pressure-sensitive diaphragm, or force transducer. In the embodiment wherein the sensor device 100 is an implantable cardiac sensor, the sensor elements 107 may include structures for transducing ventricular wall motion into stiffness measurements. Other suitable sensor elements 107 may include, but are not limited to, interdigitated electrodes over flexible PVDF membranes, capacitive diaphragm-based MEMS for differential pressure sensing, and strain gauges on serpentine conductive traces for large-deformation zones.

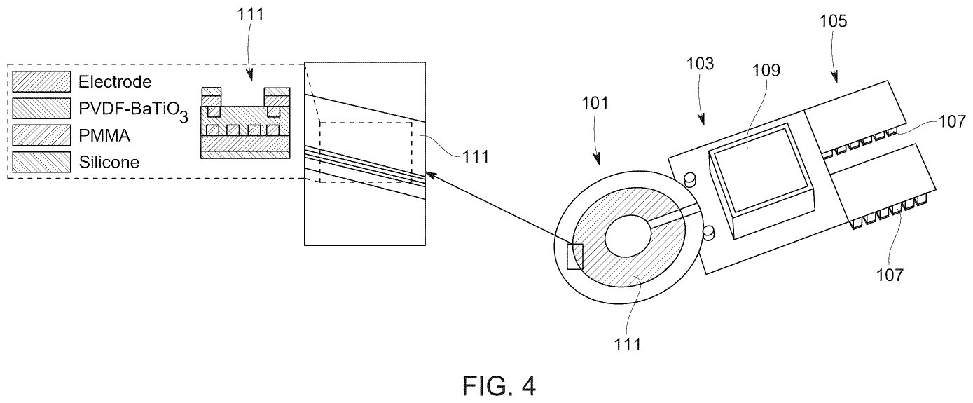

FIG. 4 schematically illustrates an enlarged view of a harvester module 101 and a power harvesting element 111 of a sensor device 100 according to an embodiment of the present disclosure. In one embodiment, harvester module 101 includes a power harvesting element 111 formed with PVDF-BaTiO₃ having gold plated electrodes and a 0.01 mm of silicone coating layer. The use of polymethyl methacrylate (PMMA) enables piezoelectric design to operate at a low natural frequency and within a small area. An example of a small area includes an area of 12 mm×4 mm. The harvester element 111 includes a primary beam of PVDF-BaTiO₃, and secondary array beam with PMMA. PMMA piezoelectric nanofibres beams are placed on the PVDF-BaTiO₃ beam as shown in FIG. 4. In this embodiment, the configuration of the sensor element 107 results in two natural frequencies (1.02 Hz and 1.52 Hz) in a cut out electrode beam, which are combined to increase the overall power produced. This configuration permits a reduction in stiffness, including up to half the stiffness, while operating. In other embodiments, alternative configurations of the power harvesting element 111 may be employed. For example, a multilayer stacked architecture of PVDF and ceramic-based materials (e.g., BaTiO₃ or PZT, optionally lead-free variants) may be used to amplify charge density. Alternatively, a cantilever configuration with a proof mass attached to the free end may be implemented to tune the resonance of the harvester module to match low-frequency biological motion. Triboelectric components—such as layers of PTFE and nylon with microstructured surface topology-may also be integrated to work synergistically with the piezoelectric components, producing high-voltage spikes from contact-separation cycles. Other suitable triboelectric configurations may include contact-separation or sliding triboelectric layers (e.g., PTFE-Nylon interfaces); microstructured or nanopatterned surfaces to increase surface area and/or floating center electrode suspended by dielectric layer to harvest peak voltages. Additional examples of arrangements for power harvesting element 111 include, but are not limited to, a cantilever beam with tip mass to lower resonant frequency, a multilayer piezo stack for improved voltage gain and/or a hybrid triboelectric-piezoelectric system with stacked electrode layers. In one embodiment where the sensor device 100 is positioned on a heart, the harvester module 101 generates between about 56 μW and 150 μW from cardiac motion under physiological operating conditions

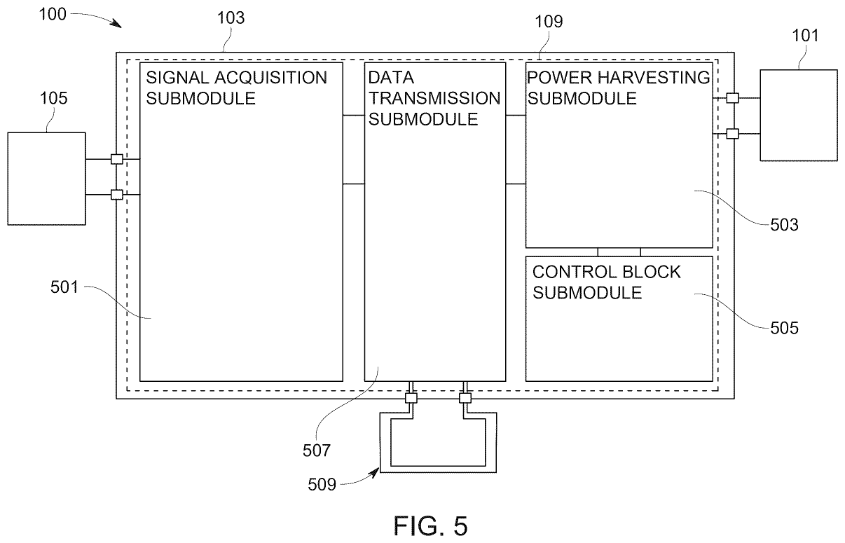

FIG. 5 schematically illustrates an enlarged view of controller module 103 according to an embodiment of the present disclosure. The controller module 103, as shown in FIG. 5, includes circuitry 109 having a signal acquisition submodule 501, a power harvesting submodule 503, a control block submodule 505, a data transmission submodule 507 and an antenna 509 operably connected. To be operably connected, the components are electrically connected in a manner that permits the components to receive signals from the sensor module 105, receive power from power harvesting module 101 and send an output signal 203. In embodiments wherein the sensor device 100 is to be implanted, the controller module 103 may be coated or otherwise encapsulated to seal the components of the controller module 103 to render the component biocompatible. Suitable materials for encapsulation may include, for example, medical grade silicon or any other suitable medical grade encapsulation material.

### Signal Acquisition Submodule 501

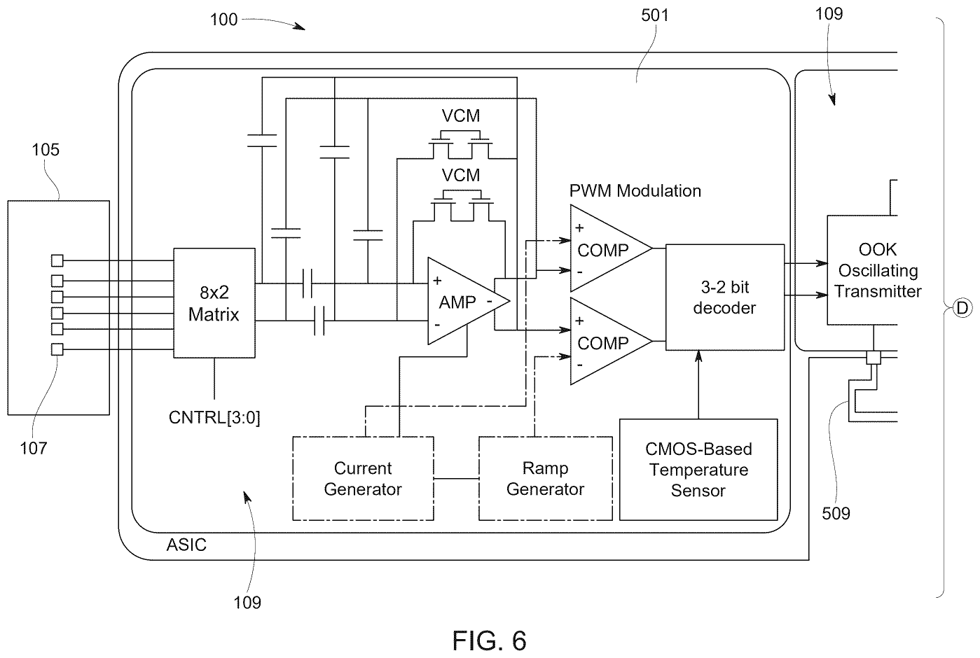

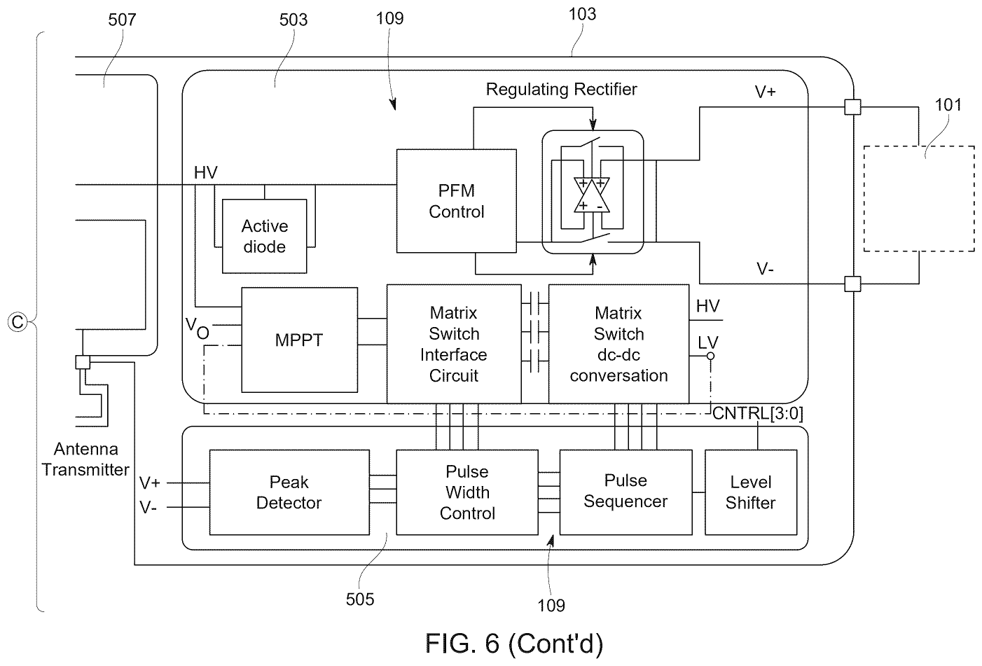

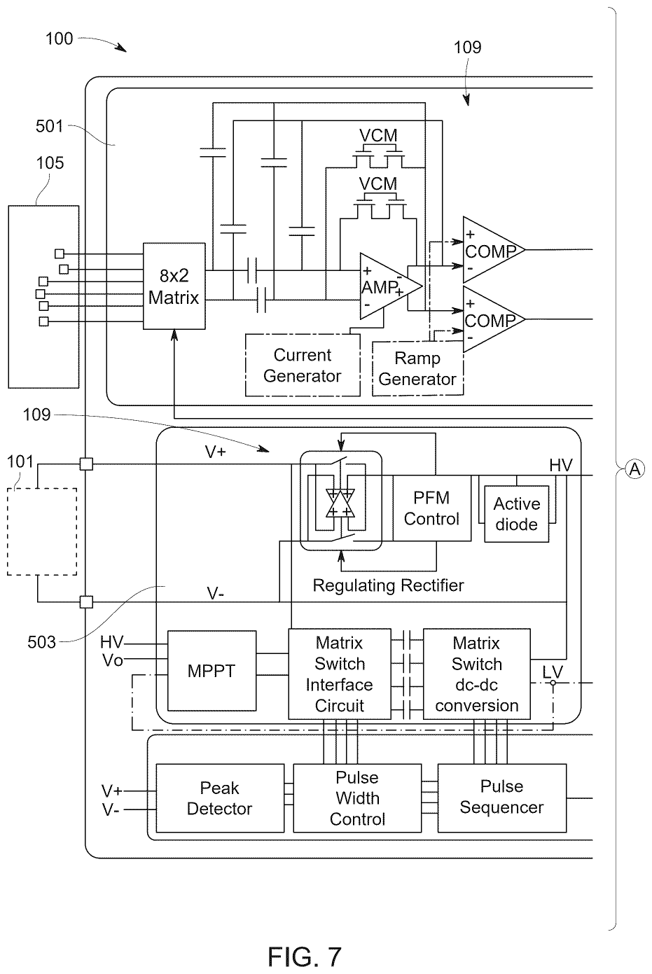

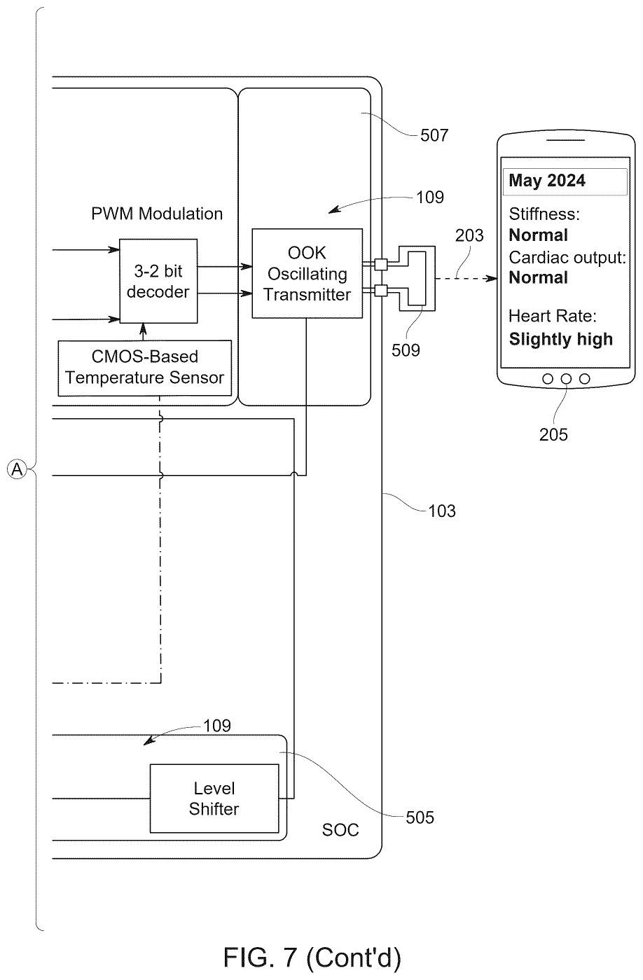

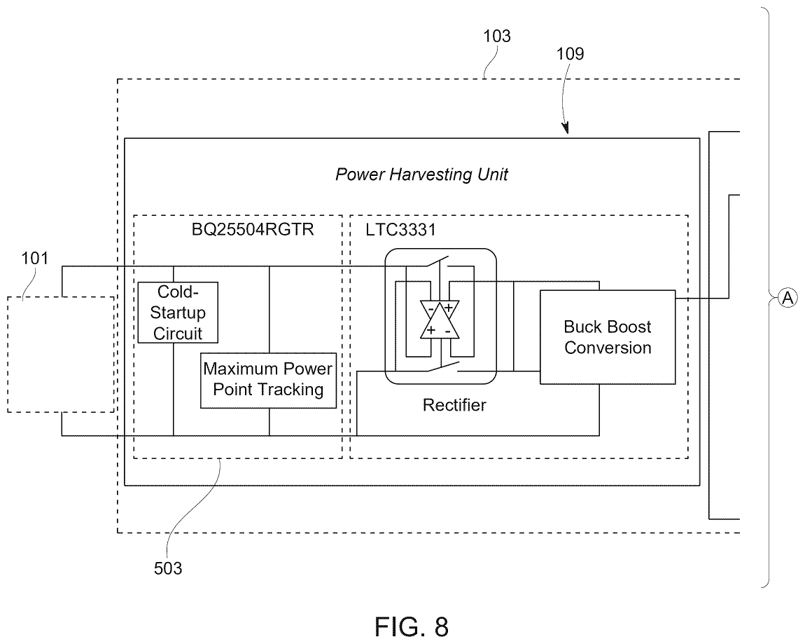

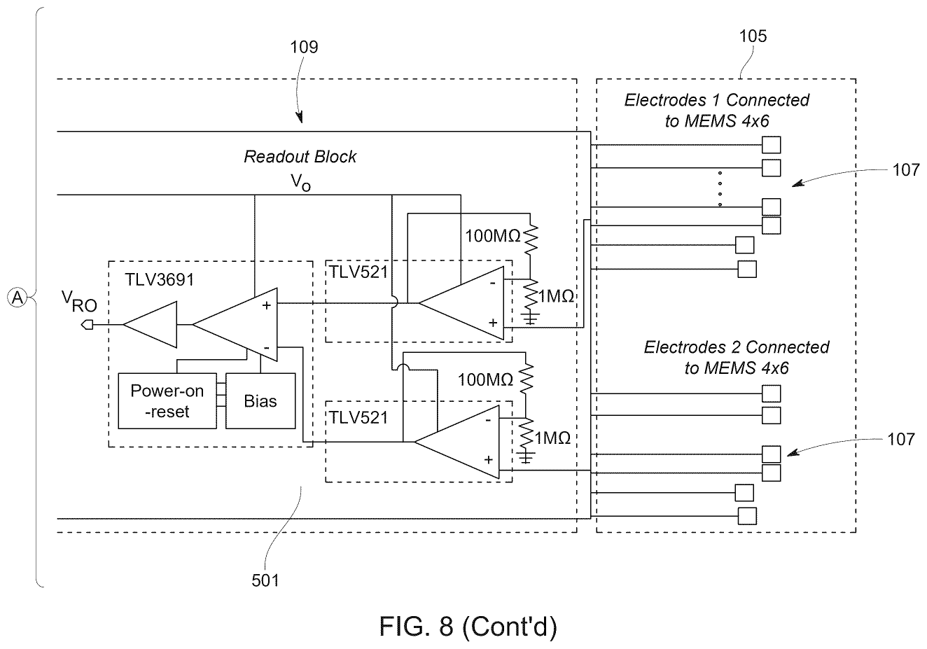

The signal acquisition submodule 501 and may include circuitry 109 having components suitable for acquiring, measuring and/or detecting signals from sensor elements 107 of sensor module 105. For example, in one embodiment, circuitry may include circuitry a maximum power point tracking (MPPT) control circuitry, a capacitively coupled amplifier, a CMOS-based temperature sensor, and an On-Off Keying (OOK) oscillating transmitter operably connected to a dual-loop antenna. The MPPT control circuitry may operate in conjunction with synchronized switching harvesting on capacitors (Hybrid-SSHC) and PFM-controlled regulating rectifiers. In another embodiment, circuitry 109 includes a signal acquisition submodule 501 operable to process biomechanical sensor signals and extract physiological cardiac metrics. In another embodiment, circuitry 109 may include a capacitively coupled amplifier operably connected to the sensor module 105 for ultra-low noise signal amplification. In still another embodiment, as shown in FIGS. 6 and 7, the circuitry 109 for signal acquisition submodule 501 may include a capacitively coupled amplifier and complementary metal-oxide-semiconductor (CMOS)-based temperature sensor. In another embodiment, the signal acquisition submodule 501 may include, for example, multiple sensing channels configured to collect spatially distributed measurements for ejection fraction, cardiac output and stiffness determination. The components are capable of operating within the strict power constraints imposed by sub-microwatt harvested energy availability obtained from the power harvesting submodule 503, allowing continuous sensing, processing, and telemetry within a fully autonomous implantable platform. In another embodiment, as shown in FIG. 8, circuitry 109 for signal acquisition submodule 501 may include two ultra-low power TLV521 NanoPower operational amplifiers consuming approximately 595 nW each, with input-referred noise below 300 nV/√Hz, to provide high sensitivity signal amplification from piezoelectric MEMS sensors. Amplified signals from two spatially distinct sensing sites are processed via a TLV3691 hysteresis comparator, consuming approximately 255 nW, to detect myocardial deformation and derive stiffness-related metrics. This architecture enables real-time multi-site biomechanical assessment while maintaining total readout power within sub-microwatt operational limits. The multi-site sensing advantageously enables spatial mapping and the inclusion of subthreshold biasing techniques allow for further power reduction. However, the disclosure is not limited to these components for use in the signal acquisition submodule 501 and may include any components suitable for acquiring, measuring and/or detecting signals from sensor elements 107 of sensor module 105.

### Power Harvesting Submodule 503

The power harvesting submodule 503 includes circuitry 109 to harvest and condition the power sufficiently to power the signal acquisition submodule 501, control block submodule 505, and data transmission submodule 507. In one embodiment, circuitry 109 includes a power harvesting submodule 503 having a maximum power point tracking (MPPT) control circuit and power conversion circuitry configured to regulate harvested electrical energy for continuous system operation at ultra-low power levels. In one embodiment, as shown in FIGS. 6 and 7, an application-specific integrated circuit (ASIC) architecture that integrates a power harvesting submodule 503 comprising maximum power point tracking (MPPT), synchronized switching harvesting on capacitors (Hybrid-SSHC), and PFM-controlled rectifier circuits, dynamically regulating highly variable harvested energy from cardiac motion. The MPPT and Hybrid-SSHC configuration advantageously enables real-time power adaptation to variability in heartbeat amplitude/frequency. In another embodiment, as shown in FIG. 8, the power harvesting submodule 503 in the controller module 103 may include commercial maximum power point tracking (MPPT) control circuit (BQ25504RGTR) and energy harvesting power management unit (LTC3331). In this embodiment, the overall power consumption is about 2 μW. The integrated chip architecture of circuitry 109 of the power harvesting submodule 503 substantially amplifies harvested piezoelectric energy. In one embodiment, the power harvested is amplified by a factor of about 10. The amplification is provided through power conversion circuits that regulate output to supply continuous power to signal acquisition submodule 501. This permits, for example, real-time measurement of ejection fraction, cardiac output, myocardial stiffness, and tissue temperature measurements. However, the disclosure is not limited to these components for use in the power harvesting submodule 503 and may include any components suitable for harvesting power from piezoelectric sensors.

### Control Block Submodule 505

The control block submodule 505 may include circuitry 109 that provide control for the other components of the controller module 103, including the signal acquisition submodule 501, power harvesting submodule 503, and data transmission submodule 507. While control block submodule 505 is shown as a separate component in FIG. 5, the control block submodule 505 may be omitted or otherwise integrated into other components, such the harvester module 101, the sensor module 105, the signal acquisition submodule 501, the power harvesting submodule 503, and/or the data transmission submodule 507 to provide control. In one embodiment, as shown in FIGS. 6 and 7, control block submodule 505 includes a heartbeat-synchronized clock architecture enabling adaptive power allocation and dynamic energy gating based on real-time energy availability. However, the disclosure is not limited to these components for use in the control block submodule 505 and may include any components suitable for control of the various elements of the controller module 103.

### Data Transmission Submodule 507

The data transmission submodule 507 may include circuitry 109 that collects a signal, corresponding to data collected by sensor element 107 and generates an output signal 203. In one embodiment, circuitry 109 includes a data transmission submodule 507 comprising an On-Off Keying (OOK) transmitter, configured to wirelessly transmit acquired cardiac data to an external receiver. While not so limited, the OOK may operate in the 2.45 GHz ISM band. In another embodiment, the data transmission submodule achieves energy efficiency of at least 5 picojoules per bit at data transmission rates of up to 10 Mbps while radiating at −33 dBm or less. In one embodiment, the data transmission submodule is capable of sending an output signal 203 via antenna 509 at implantation depths of at least 4 cm or from 4 to 6 cm.

In one embodiment, as shown in FIG. 7, the output signal 203 may be wireless signal sent via antenna 509. FIG. 7 shows signal receiver 205, which receives output signal 203. In one embodiment, as shown in FIG. 7, the circuitry 109 of data transmission submodule 507 may include On-Off Keying (OOK) modulation, for example, at 2.45 GHz. In this embodiment, the data transmission submodule 507 achieves an energy efficiency of 5 pJ/bit with a data transmission rate of 10 Mbps while radiating at −33 dBm, setting a new benchmark for performance in this field. In one embodiment wherein the sensor device 100 is an implanted device on the heart, this transmission architecture allows real-time data communication via output signal 203 to be continually provided directly from cardiac energy harvested by power harvesting element 111, overcoming prior limitations where implantable telemetry required external power supplementation or operated only intermittently due to power constraints. The system enables real-time local monitoring of myocardial stiffness with up to and including 96.7% accuracy, providing continuous disease progression tracking outside intensive care environments. In one embodiment, the total system power consumption is constrained to approximately up to and including 100 nW or about 98.8 nW, permitting fully autonomous operation solely on mechanical energy harvested from, for example, cardiac motion. The integrated telemetry unit transmits data regarding sensed conditions externally without requiring any external power source or battery support. The sensor device 100 operates solely from the mechanical energy derived from heartbeat motion, achieving complete implantable autonomy through ultra-efficient power harvesting, management, and utilization.

Examples of commercial applications for the sensor device 100 according to the present disclosure include but are not limited to 1) an implantable heart sensor, providing up-to-date data on the current condition of the heart, including muscle stiffness and cardiac flow; 2) add-on components to enhance existing medical devices. These components can address gaps in current medical device technology, such as the lack of a self-powered system, wireless transmission capabilities, or the ability to sense cardiac ejection fraction, muscle stiffness and cardiac output accurately; 3) other medical applications requiring self-powered remote sensing; and 4) non-medical uses, such as measurements in industrial, transportation, construction, power system, consumer applications requiring self-powered remote sensing.

In one embodiment according to the disclosure, the sensor device 100 harvests energy and senses one or more conditions from a target substrate 201, such as the myocardial surface of a heart. In this embodiment, the sensor device is implanted on the heart in a location where the heartbeat induces a strain on the power harvesting element 111 of the harvester module 101, which provides power to controller module 103, which utilizes sensor elements 107 of sensor module 105 to measure changes in the heart geometry. The data obtained from the sensed conditions may be utilized, for example, for early detection of cardiovascular allograft vasculopathy (CAV). The sensor array size and distribution of the sensor elements 107 may be arranged to optimize the sensitivity to changes in the environment. Optimization may be based upon, for example, placement over left ventricle wall, a beam length and thickness tuned to 0.6-1.8 Hz and maximizing strain-coupling by curvature matching between tissue and device. For example, the sensor device 100 may be placed at the lower left ventricle of the heart, as this location yields the highest power output. Furthermore, the constriction of blood vessels frequently manifests in the context of smaller vasculature, thereby requiring invasive means to detect and monitor.

In one embodiment, the sensor device 100 according to the present disclosure may include a biocompatible, implantable flexible carrier 113. Suitable sizes for the flexible include, for example, in a range from approximately 10 mm×5 mm to 30 mm×20 mm, depending on application and implant location. In other embodiments, the flexible carrier 113 may be configured for epicardial, pericardial, or endocardial implantation depending on clinical requirements. The harvester module 101 generates between about 10 μW and 150 μW. For example, harvest module 101 may generate at least about 56 pW of electrical power from myocardial motion, sufficient to power all integrated system functions. In addition, the sensor device 100 includes a controller module 103 having an integrated system-on-chip (SoC) comprising power management, signal acquisition, signal processing, and wireless telemetry circuits, including a wireless telemetry module employing On-Off Keying (OOK) modulation at 2.45 GHz ISM band, configured for ultra-low power data transmission. The sensor device 100 further includes sensor module 107 configured for real-time biomechanical assessment of cardiac stiffness and output with approximately 96.7% measurement accuracy.

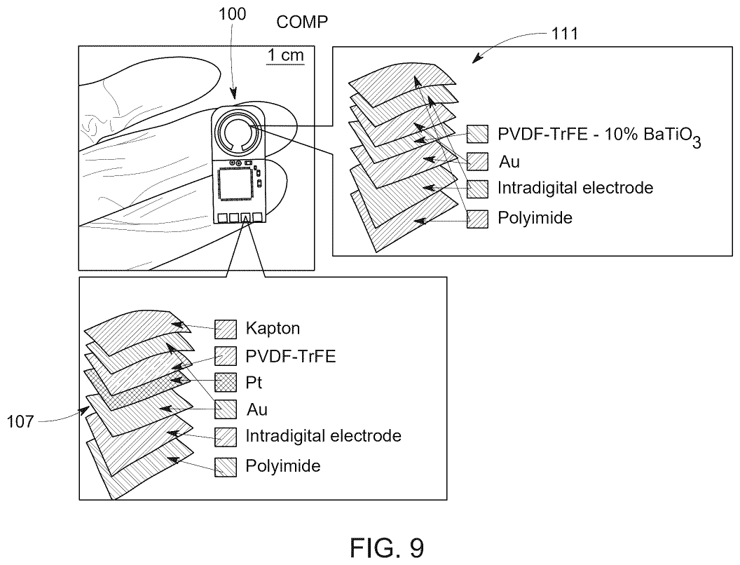

FIG. 9 schematically illustrates a sensor device 100 having an enlarged view of a sensor element 103 and an enlarged power harvesting element 111 according to another embodiment of the present disclosure. As shown in FIG. 9, the sensor element 107 includes a layered system made up of polyimide/an intradigital electrode/gold/platinum/PVDF-TrFE/gold/Kapton in an arrangement that is capable of producing a signal in response to a condition. Likewise, FIG. 9 shows a power harvesting element 111 having a layered system made up of polyimide/intradigital electrode/gold/PVDF-TrFE-10% BaTiO₃/intradigital electrode/polyimide in an arrangement that is capable of generating power from an induced strain. Although the specific layered arrangement may be used together, as shown in FIG. 9, the present disclosure is not so limited and may include one of the layered arrangements with a different arrangement of power harvesting element 111 or sensor element 103.

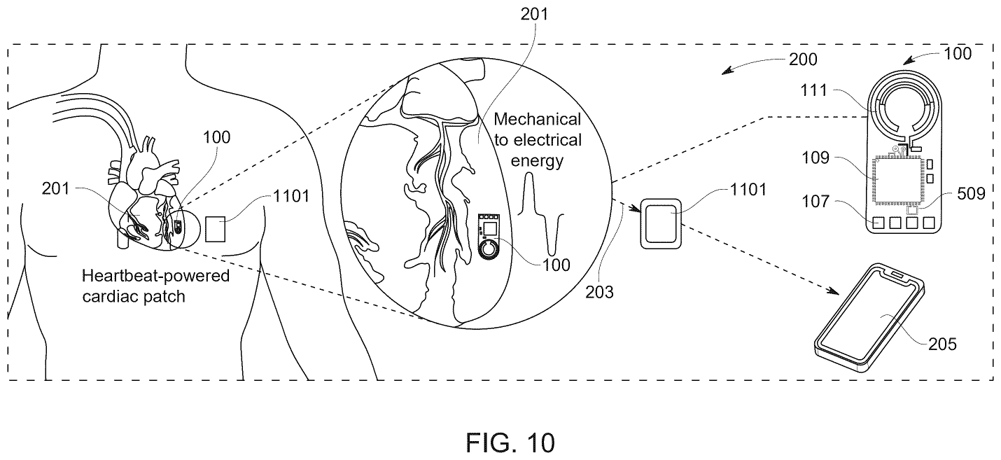

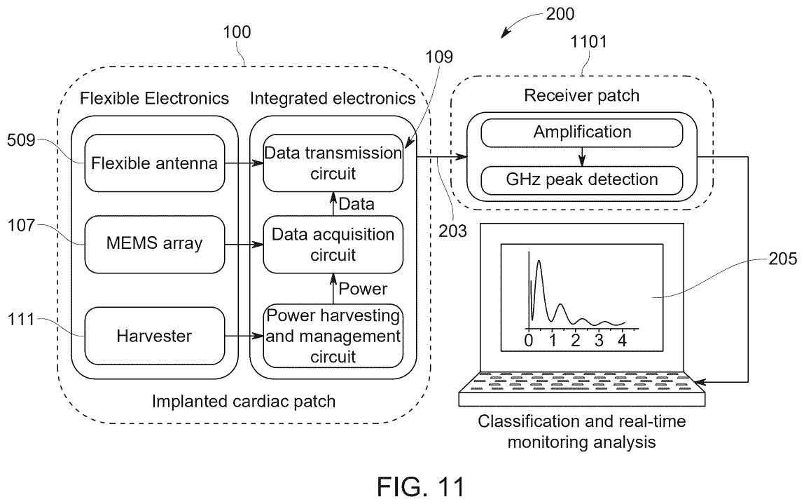

FIG. 10 schematically illustrates a monitoring system 200 including a receiver patch 1101 according to another embodiment of the present disclosure. The monitoring system 200 includes the elements and operates essentially as shown and described above with respect to FIG. 2. However, the monitoring system 200 of FIG. 10 further includes a receiver patch 1101, which provides circuitry 109 that receives the output signal 203 and amplifies the signal prior to sending to the signal receiver 205. The arrangement including the receiver patch 1101 permits operation where the signal receiver 205 may be provided a greater distance or more remotely from the sensor device 100. For example, FIG. 11 schematically illustrates the signal and data flow within the monitoring system of FIG. 10. The process shown in FIG. 11 results in an output signal generated with power from power harvesting element 111, a signal from sensor element 107 and with power and data processing with circuitry 109. The receiver patch 1101 amplifies the signal and sends the signal to the signal receiver 205. The amplification may be provided with, for example, circuitry 109 as shown and described above with respect to controller module 103 for amplification of the signal.

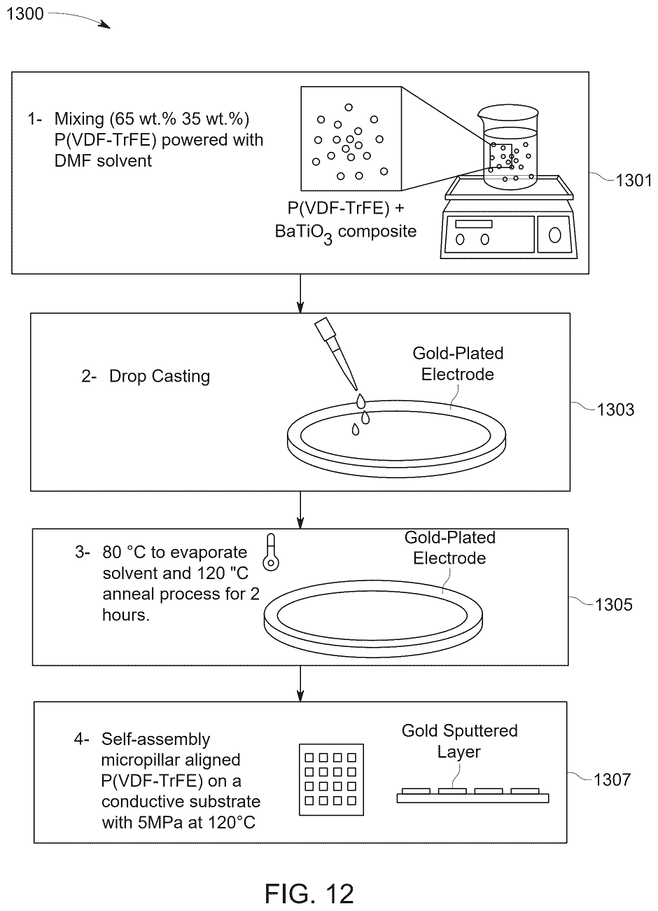

FIG. 12 is a flow chart illustrating a method 1300 for fabricating a sensor element 107 according to an embodiment of the present disclosure. Method 1300 forms a sensor element 107 including a fibrous membrane of PVDF-BaTiO₃. The sensor's piezoelectric fibrous membrane PVDF-BaTiO₃ is fabricated using an electrospinning technique. Deposition thickness depends on electrospinning duration, producing poling effects to enhance piezoelectricity of nm-fibers. The size of the fabricated sensing electrode is configured to achieve a low input referred noise (IRN)

$$IRN = 20 {log}_{10} ( \frac{MDP}{20 μ Pa} ) \qquad ( 7 )$$

where MDP is the minimal detectable pressure. The design is optimized to remove pressure cancellation due to diffraction to reduce sensitivity. The IRN and sensitivity are dependent on the size of MEMS electrode, the material properties, readout circuitry, and sensor's environmental conditions. The optimized size targeted IRN is less than 25 (dB). The sensitivity of the sensor is calculated through bending analysis with a natural frequency of 1.33 Hz and a displacement of 4.5 nm. A composite of poly(vinylidene fluoride-co-trifluoroethylene) (P (VDF-ReFE))+BaTiO₃ is formed by mixing P(VDF-ReFE) and BaTiO₃ in dimethylformamide (DMF) solvent (step 1301). The composite mixture formed is drop cast on a gold-plated electrode (step 1303). The deposited composite mixture is dried and annealed (step 1305). Annealing may be provided at a temperature of from about 120 to about 150° C. Additional layers are provided to the annealed composite mixture of PVDF and BaTiO₃ to form sensor element 107 (step 1307).

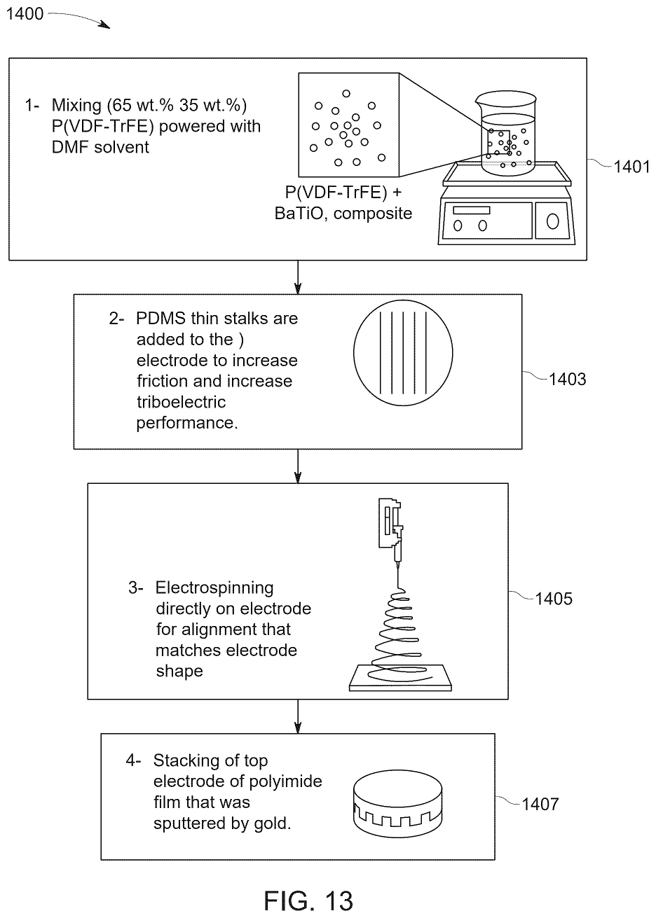

FIG. 13 is a flow chart illustrating a method 1400 for fabricating a power harvesting element 111 according to an embodiment of the present disclosure. A composite of poly(vinylidene fluoride-co-trifluoroethylene) (P(VDF-ReFE))+BaTiO₃ is formed by mixing P(VDF-ReFE) and BaTiO₃ in dimethylformamide (DMF) solvent (step 1401). Polydimethylsiloxane (PDMS) or polymethyl methacrylate (PMMA) stalks are added to a gold-plated electrode (step 1403). The stalks are added to increase friction and increase triboelectric performance of the power harvesting element 141. The P(VDF-ReFE) and BaTiO₃ composite material is electrospun on the electrode and stalks in an arrangement that matches the electrode shape (step 1405). The deposited composite material may be adjusted utilizing electrospinning parameters (e.g., voltage, tip-collector distance) to provide the desired layer properties. A top electrode of polyimide and sputtered gold is applied to the PVDF and BaTiO₃ formed from the electrospun P(VDF-ReFE) and BaTiO₃ layer. PDMS stalks may be deposited to enhance triboelectric friction.

### EXAMPLE

One example of sensor device 100 is a biocompatible, in-body energy harvesting piezoelectric biosensing device utilizing sensor elements 107 for the detection of cardiac allograft vasculopathy. This system includes an ultralow power readout circuit that operates solely from power harvested from heartbeats. With a natural frequency of 1.52 Hz, the nonlinear piezoelectric harvester generates 56 μW in ex-vivo testing. The device has a 4×6 array of fabricated MEMS sensors, which provides over 80% accuracy in detection of heart geometry changes.

The sensor device 100 according to this Example addresses several challenges: 1) Dimension optimization to adjust the resonance of the piezoelectric harvester to match the heartbeat frequency of 0.6-1.80 Hz. When damping effects are small, the derived resonant frequency f₀ of the system is:

$${f}_{0} = \frac{1}{2 π} \sqrt{\frac{3 E \cdot {T}^{3} / 4 {L}^{3} \cdot ( 1 - {v}^{2} )}{{m}_{eff} + {C}_{P} \cdot {C}_{e}}} \qquad ( 1 )$$

where E, T, L, υ, m_{eff}, Cₚ, Cₑ are elastic modulus, thickness, length, Poisson's ratio of the piezoelectric material, its effective mass, material capacitance, and electrical capacitance of circuitry, respectively. Tand L are adjusted to provide optimal power excitation. 2) Optimum transmission of the harvested power by matching the internal capacitance of the piezoelectric harvester with the external receiver circuit, by using ultra-low power electronics to ensure maximum power transfer at low power consumption. 3) Monitoring the geometry of the vessels using an array of PEH for early detection of CAV and operated at nW power levels provided exclusively by the power harvesting element 111. 4) Conditioning power resulting from heartbeats that are an unstable and low frequency power into a stable power output utilizing circuitry 109 that smooths the variability in power output. 5) Miniaturization and good flexibility are provided so that the device may be placed on the surface of the heart.

### Modelling Analysis of Piezoelectric Devices

The design was simulated with COMSOL Multiphysics 6.0 (COMSOL Inc.). Solid mechanics and pressure acoustics boundary conditions with a fine mesh of 0.002 mm were used. Placement consideration of circuits for performance optimization were taken into consideration.

When stress is applied to a PEH material it generates a voltage through a process known as ‘direct effect’. The coupling relationship between the strain and the electric field can be determined as follows:

$$S = {S}_{E} T + {d}^{T} E \qquad ( 2 )$$

$$D = dT + {ε}_{T} E \qquad ( 3 )$$

where S is the zero-to-peak strain, S_{E} is the material compliance, T is the stress, E is the electric field, D is the electric displacement field, d is the coupling properties, and ε_{T} is the permittivity. The equations of solid mechanics and electrostatics are used to analyze this problem. The equations (2) and (3) can be rearranged into the stress-charge form, which relates the material stresses to the electric field. In the frequency domain, the behavior is modelled using material properties with complex values, in the absence of the loss mechanism, as shown below:

$$S = {\tilde{S}}_{E} T + {\tilde{d}}^{T} E \qquad ( 4 )$$

$$D = \tilde{d} T + {\tilde{ε}}_{T} E \qquad ( 5 )$$

Where \tilde{S}_{E}, \tilde{d}, \tilde{ε} are complex matrices including an imaginary part used to define the dissipative function of the material. The real and imaginary parts of the material are defined to account for the symmetrical properties of the modelled material. Mechanical damping is introduced in the system by computing several eigenfrequencies, using different sets of boundary conditions. All the eigenfrequencies must be positive imaginary to represent a damped motion, while considering mechanical, coupling, and conduction losses within the system. The placement of the device is carefully chosen to minimize its impact on the heart's function when applied to the surface.

The generated piezoelectric signal power was found to be highly dependent on material thickness, electrode geometry, and strain distribution. The system design optimizes these parameters to simultaneously maximize sensitivity to myocardial deformation while ensuring sufficient electrical output to support sub-microwatt system operation. This dimensional optimization directly supports reliable biomechanical signal acquisition under extreme energy constraints inherent to fully self-powered implantable systems. When the device is subjected to pressure, the generated power is dependent on piezoelectric layer thickness. The optimum power, Pᵣₘₛ, is:

$${P}_{rms} = \frac{ω {T ( {V}_{amp} {d}_{31} {sE}_{P} )}^{2}}{2 {ε}_{3 S}^{S}} \qquad ( 6 )$$

where ω is the angular frequency of vibration, T is the thickness of piezoelectric, Vₐₘₚ is the amplitude of vibration, d₃₁ is the piezoelectric constant, S is the side of the piezoelectric square, Eₚ Young's Modulus, and

$${ℰ}_{33}^{S}$$

is the permittivity constant. In (6) the side for which the power can be optimized is found to be s=180 μm. The electrodes connecting the sensors to the circuits were also modelled to find the resonance frequency, generated peak voltage, and the average power as functions of the piezoelectric thickness.

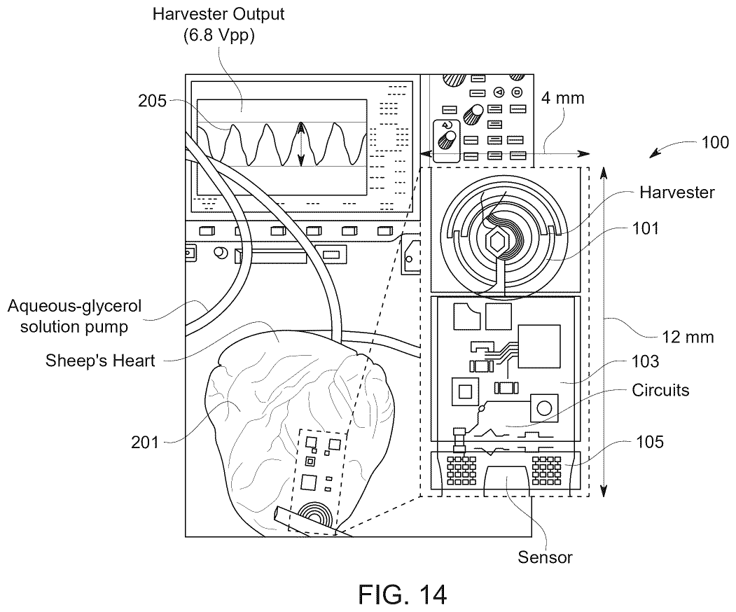

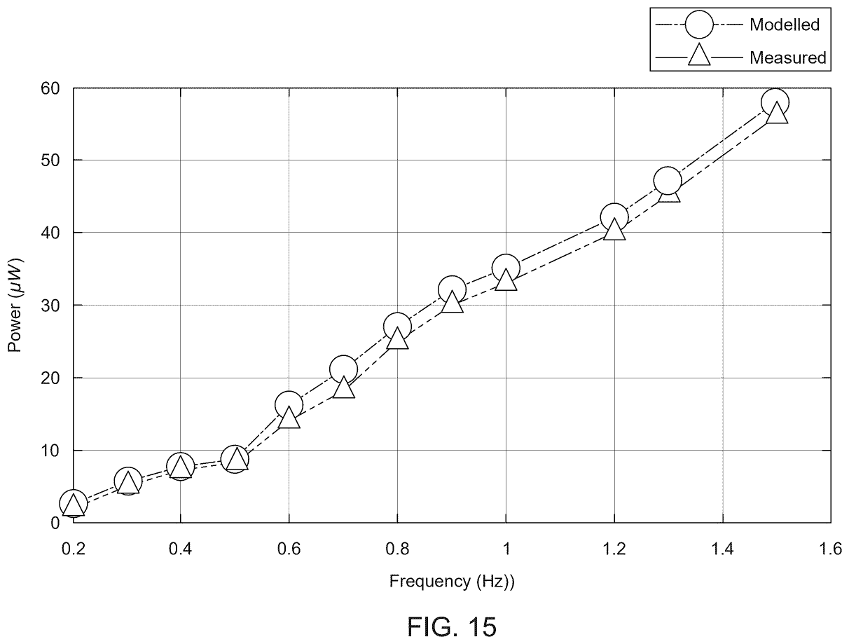

FIG. 14 shows the ex-vivo measurement setup with a sensor device 100 according to the present disclosure positioned on sheep's heart to mimic the physiological condition of a beating heart. As described above with respect to FIGS. 1, 2 and 8, the sensor device 100 includes a harvester module 101, a controller module 103 and a sensor module 105. The heart was perfused at 1 Hz with an aqueous-glycerol solution containing 10% glycerol, which had a viscosity of 5 centipoise (cP), using a perfusion pump. This solution was chosen due to its similarity in viscosity to blood. The pump cycle causes heart movement mimicking the natural heartbeat.

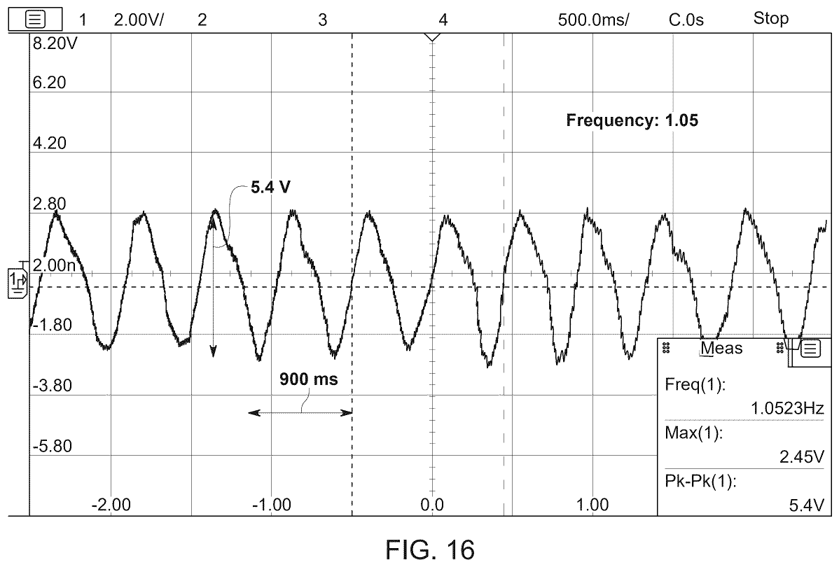

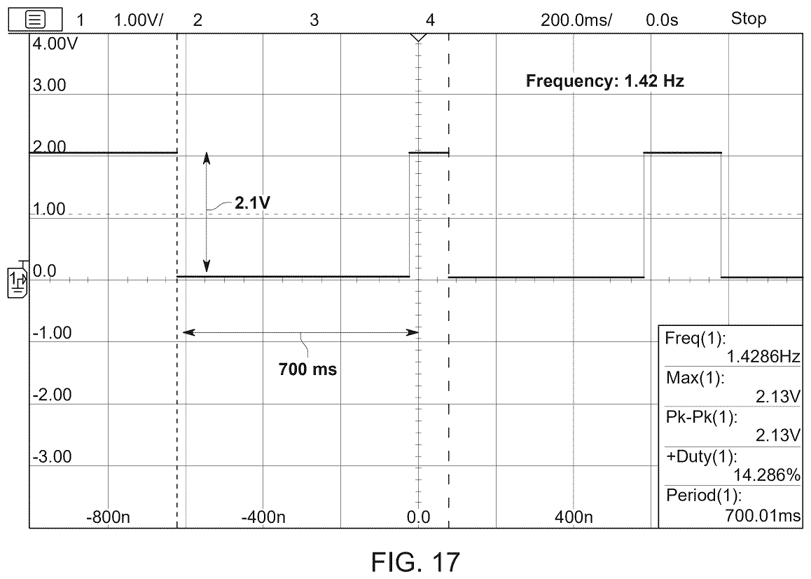

Tables 1 and 2 show a comparison with the state-of-the-art cardiac harvesters. FIG. 16 is a graph showing measured and modelled power output for a 1 MΩ resistive load over a range of heartbeat frequencies from the measurement setup in FIG. 14. The bandwidth of the power harvester module 101 is widened by reducing its sensitivity to vibration in the excitation by using a nonlinear energy harvester which avoids a shift in the excitation frequency from the resonance frequency. The power levels can vary at given excitation parameters but remain in a controllable range due to the power harvesting circuitry of controller module 103. FIG. 16 shows the measured output voltage waveform at 1.02 Hz produced by the harvester module 101 and the rectified voltage, V₀. The readout block voltage output, V_{RO}, is a square waveform because of pressure applied to the MEMS as shown in FIG. 17.

TABLE 1

| Testing Setup | In-Vitro | In-Vivo | In-Vivo | Ex-Vivo |
|---|---|---|---|---|
| V_{O (Peak)} | Comp. Ex. 1 | Comp. Ex. 2 | Comp. Ex. 3 | Example 1 |
| R_{load} | NA | 10.8 MΩ | NA | 1 MΩ |
| P_{out, Max} | 32 μW | 1 μW | NA | 56 μW |
| MPPT | No | No | No | Yes |
| Biocompatible | No | No | No | Yes |

TABLE 2

| | Comp. | Comp. | Comp. | Comp. | Exam- |
|---|---|---|---|---|---|
| | Ex 3 | Ex. 4 | Ex. 5 | Ex. 6 | ple 2 |
| Energy Harvesting | RF | Glucose | RF | Resonator | Heart- |
| Source | | | | | heat |
| Real-time | No | No | No | No | Yes |
| Tx Frequency | 412 | 920 | 457.5 | 2400 | 2450 |
| (MHz) | | | | | |
| Tx Modulation | OOK | OOK | OOK | OOK/FSK | OOK |
| TX POUT (dBm) | −19.7 | −52.89 | −22 | −33 | −6.2 |
| Data Rate (bps) | 0.15 | 5k | 5M | 10M | 10M |
| Power Active (W) | 1.14m | N/A | 191μ | 70μ | 70n |
| Power Leakage (W) | N/A | N/A | N/A | 128p | 24.3p |

The sensing provided by the Example according to the present disclosure is suitable for early detection of CAV for post heart transplantation patients. The fabricated sensor device 100 operates at heartbeat frequencies with sufficient power harvesting and sensing. The device according to the Example provides 56 μW at a heartbeat rhythm of 1.52 Hz, sufficient for powering MEMS sensors detecting CAV, and a sub-nW communication module. FIG. 17 is a graph showing a measured voltage response from the signal acquisition submodule 501 of controller module 103 from the measurement setup in FIG. 14.

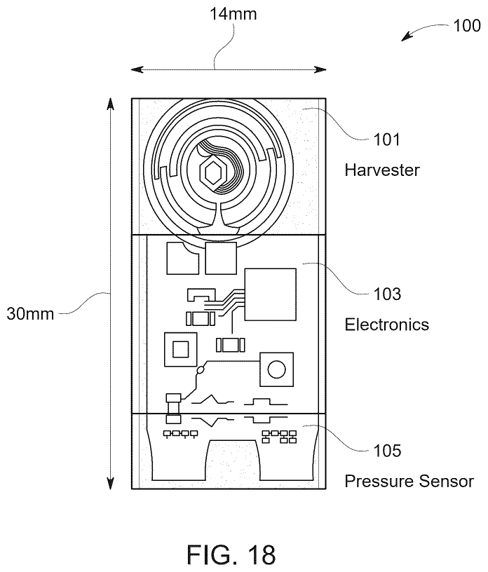

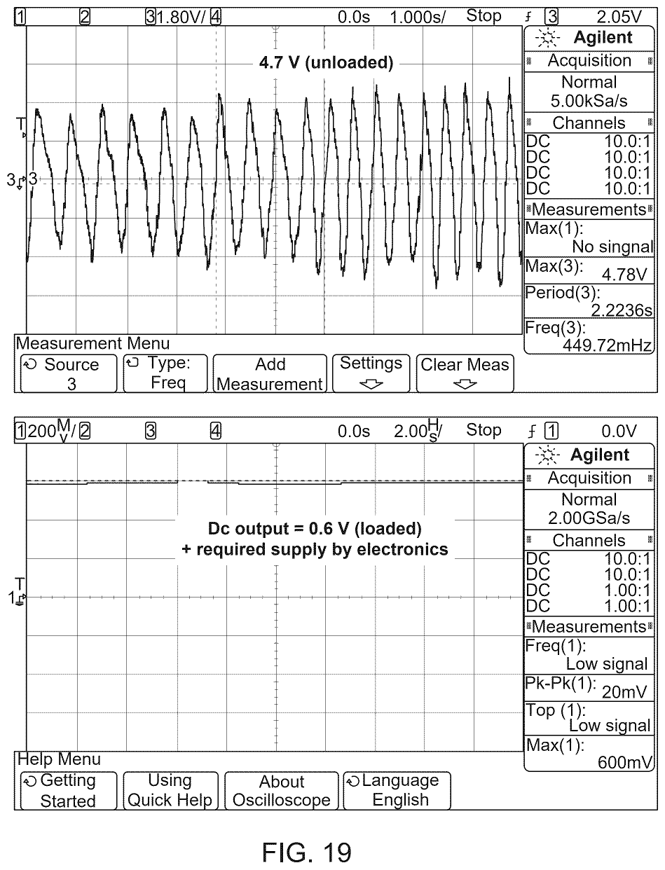

FIG. 18 shows an exemplary configuration of a sensor device 100 according to the present disclosure. In this exemplary configuration, the sensor device 100 includes a harvester module 101, a controller module 103 and a sensor module 105 adjacent to one another. FIG. 19 is a graph showing loaded and unloaded voltage measurements from the sensor device shown in FIG. 18.

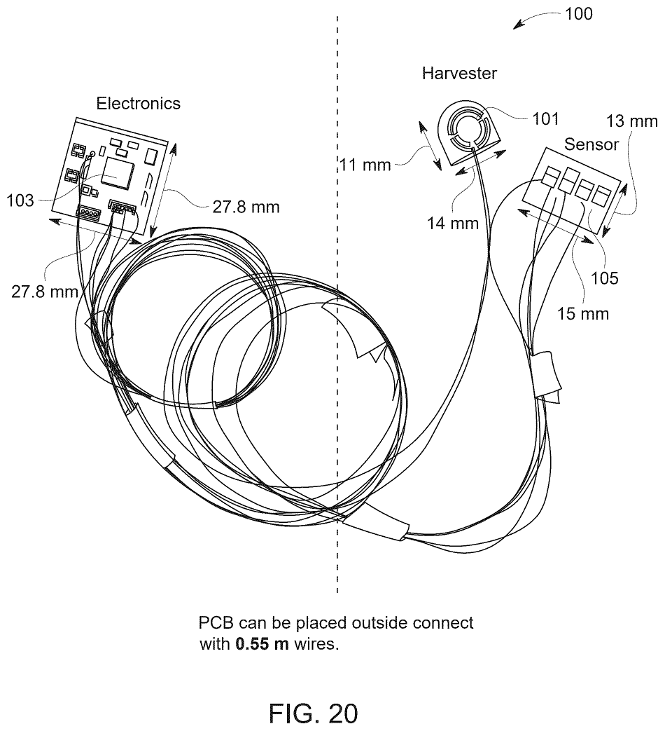

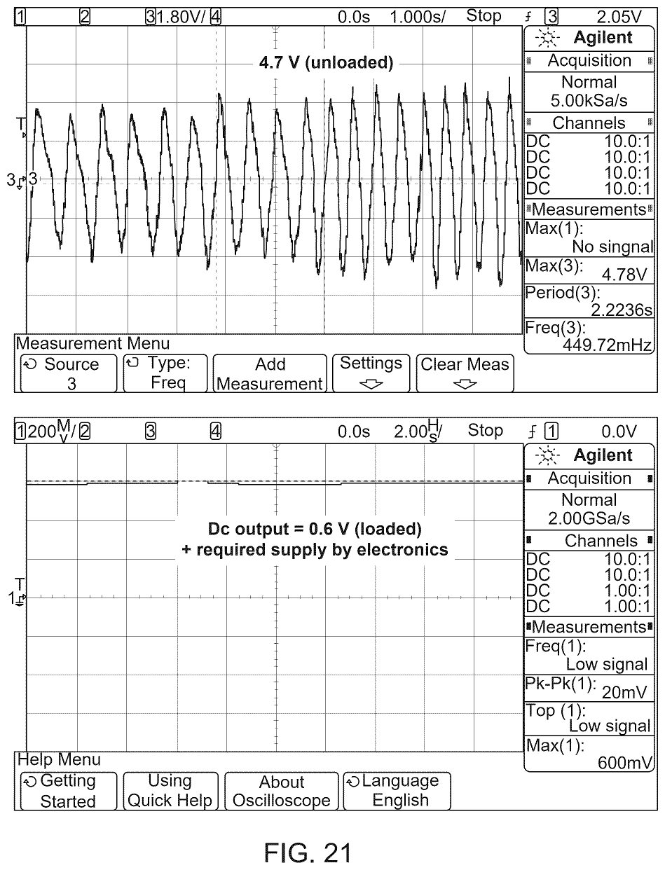

FIG. 20 shows another exemplary configuration of a sensor device according to the present disclosure. In this exemplary configuration, the sensor device 100 includes a harvester module 101, a controller module 103 and a sensor module 105, where the controller module is connected to the harvester module 101 and the sensor module 105 by wires that permit the placement of the controller module 103 outside of the body when utilized as an implant. FIG. 21 is a graph showing loaded and unloaded voltage measurements from the sensor device shown in FIG. 20.

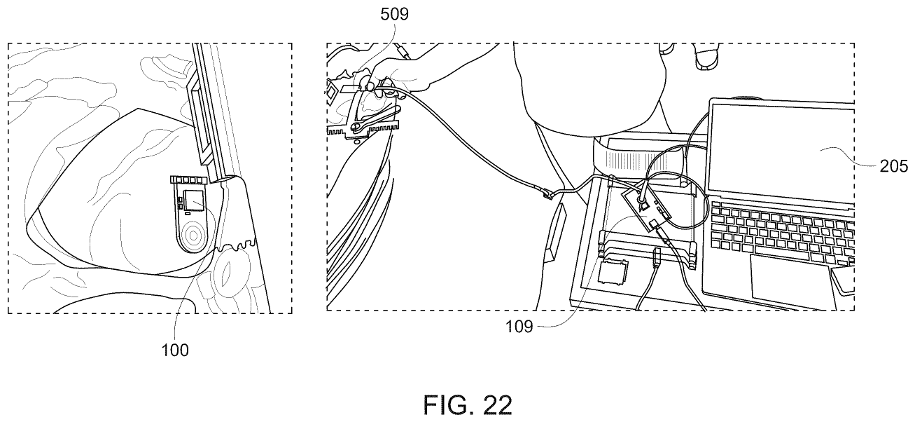

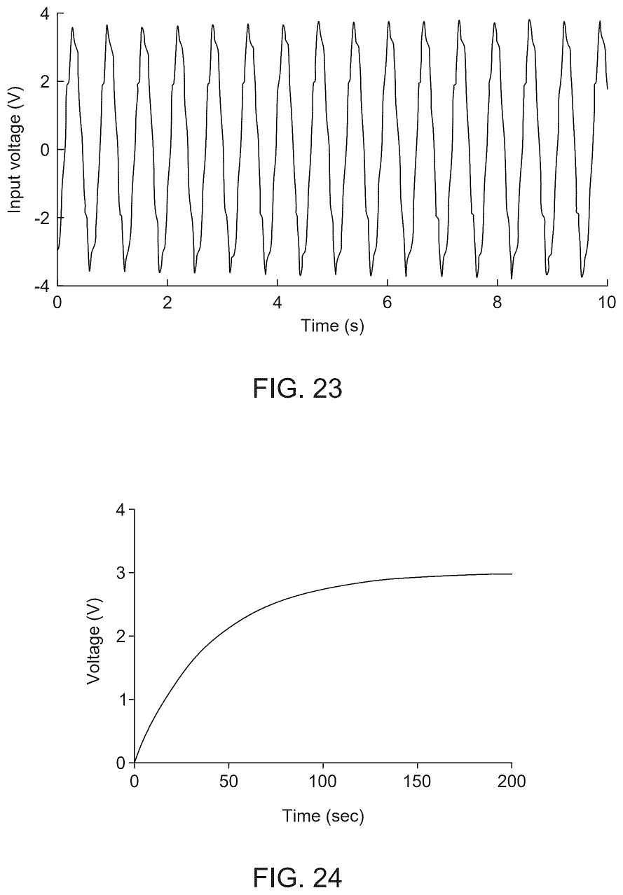

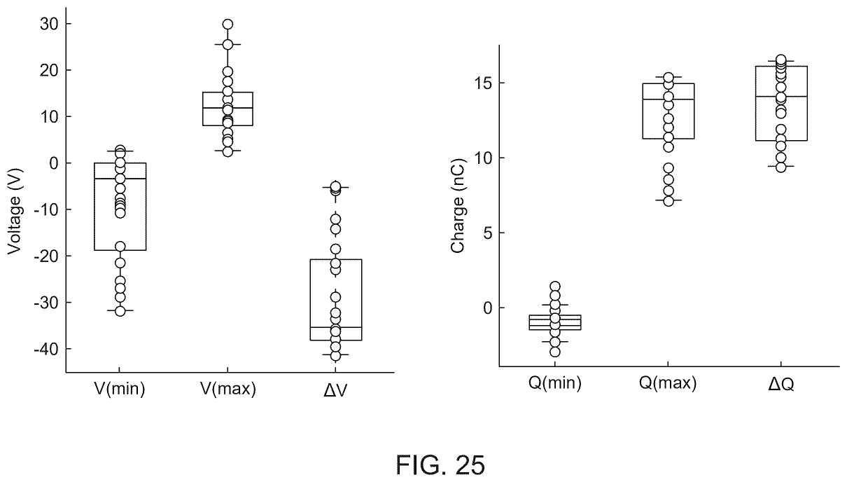

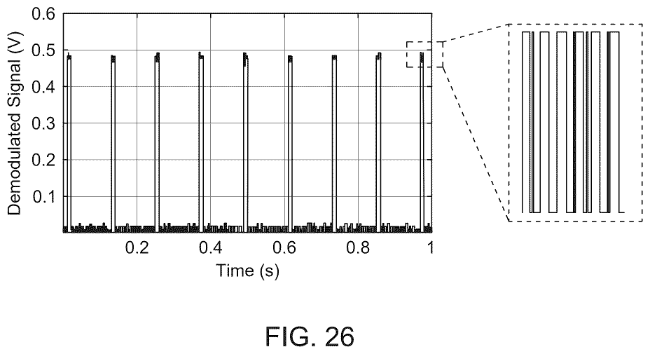

FIG. 22 shows the in-vivo measurement setup with a sensor device 100 according to the present disclosure. The sensor device 100 operates as shown and described above. Data was obtained from the measurement setup shown in FIG. 22 and are displayed in FIGS. 23-27. FIG. 23 is a graph showing time-domain signal output from the implanted sensor device 100 in the measurement setup in FIG. 22. The time-domain signal output from the implanted patch was measured with a 50 Ω load and connected via GHz transmission line during in-vivo cardiac cycles. The data demonstrates signal stability and repeatability with each heartbeat, confirming successful power harvesting and pressure-induced signal modulation. FIG. 24 is a graph showing frequency-domain spectrum of the received signal from the implanted sensor device 100 after amplification and peak detection using GHz board and antenna in the measurement setup in FIG. 22. The clear peaks shown corresponding to cardiac motion frequencies, validate that the transmitted signal carries frequency-domain information representative of cardiac dynamics. FIG. 25 is a graph showing signal amplitude comparison from the implanted sensor device 100 between loaded and unloaded conditions (receiver present vs. open-circuit) in the measurement setup in FIG. 22. The data confirms that energy is effectively harvested and utilized by the integrated electronics to power wireless transmission-loaded output matches expected energy budget. FIG. 26 is a graph showing peak analysis and tracking over time to detect cardiac anomalies from the implanted sensor device 100 in the measurement setup in FIG. 22. The data shows viability for continuous, autonomous monitoring and early event detection (e.g., arrhythmia or stiffness changes) without external power input.

While the exemplary embodiments illustrated in the figures and described herein are presently preferred, it should be understood that these embodiments are offered by way of example only. Accordingly, the present application is not limited to a particular embodiment, but extends to various modifications that nevertheless fall within the scope of the appended claims. The order or sequence of any processes or method steps may be varied or re-sequenced according to alternative embodiments.

It is important to note that the construction and arrangement of the various exemplary embodiments is illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited in the claims. For example, elements shown as integrally formed may be constructed of multiple parts or elements, the position of elements may be reversed or otherwise varied, and the nature or number of discrete elements or positions may be altered or varied. Accordingly, all such modifications are intended to be included within the scope of the present application. The order or sequence of any process or method steps may be varied or re-sequenced according to alternative embodiments. In the claims, any means-plus-function clause is intended to cover the structures described herein as performing the recited function and not only structural equivalents but also equivalent structures. Other substitutions, modifications, changes and omissions may be made in the design, operating conditions and arrangement of the exemplary embodiments without departing from the scope of the present application.

1. A self-powered sensor device comprising:
a harvester module having a power harvesting element, the power harvesting element being arranged and disposed to harvest power from induced mechanical strain;
a sensor module having a sensor element; and
a controller module having circuitry operably connected to the harvester module and the sensor module, the controller module being configured to receive power from the power harvesting element, to receive sensor signals from the sensor element, and to wirelessly communicate an output signal corresponding to the sensor signal;
wherein the controller module is powered continuously with power harvested from the power harvesting element to generate and communicate the output signal.

2. The device of claim 1, wherein the sensor device is devoid of batteries.

3. The device of claim 1, wherein the sensor device is powered only by mechanical cardiac motion.

4. The device of claim 1, wherein the harvester module, the sensor module, and the controller module are mounted on a flexible carrier.

5. The device of claim 1, wherein the power harvesting element includes one or more piezoelectric elements selected from the group consisting of poly(vinylidene fluoride) barium titanate (PVDF-BaTiO₃) fibers, poly(vinylidene fluoride) (PVDF) fibers, poly(vinylidene fluoride) (PVDF) films, polymethyl methacrylate (PMMA), lead zirconate titanate (PZT) films, and combinations thereof.

6. The device of claim 1, wherein the power harvesting element includes a hybrid triboelectric-piezoelectric system.

7. The device of claim 1, wherein the sensor element comprises at least one piezoelectric microelectromechanical system (MEMS) sensor.

8. The device of claim 1, wherein the sensor element includes a spatial sensing array.

9. The device of claim 1, wherein the circuitry includes a power harvesting submodule comprising a maximum power point tracking (MPPT) control circuit and power conversion circuitry configured to regulate harvested electrical energy for continuous system operation at ultra-low power levels.

10. The device of claim 1, wherein the circuitry includes a signal acquisition submodule operable to process biomechanical sensor signals and extract physiological cardiac metrics.

11. The device of claim 1, wherein the circuitry includes a data transmission submodule comprising an On-Off Keying (OOK) transmitter, configured to wirelessly transmit acquired cardiac data to an external receiver.

12. The device of claim 11, wherein the data transmission submodule achieves energy efficiency of at least 5 picojoules per bit at data transmission rates of up to 10 Mbps while radiating at −33 dBm or less.

13. The device of claim 1, wherein the signal acquisition submodule includes multiple sensing channels configured to collect spatially distributed measurements for one or more of ejection fraction, cardiac output or stiffness determination.

14. The device of claim 1, wherein the circuitry further comprises a capacitively coupled amplifier operably connected to the sensor module for ultra-low noise signal amplification.

15. The device of claim 1, wherein the circuitry further comprising a CMOS-based temperature sensor.

16. The device of claim 1, wherein the circuitry includes a maximum power point tracking (MPPT) control circuitry, a capacitively coupled amplifier, a CMOS-based temperature sensor, and an On-Off Keying (OOK) oscillating transmitter connected to a dual-loop antenna.

17. The device of claim 1, wherein the harvester module generates between about 10 μW and 150 μW from cardiac motion under physiological operating conditions.

18. A monitoring system comprising:
a target substrate;
a sensor device according to claim 1, the sensor device being mounted on a flexible carrier; and
a signal receiver configured to receive the output signal from the sensor device;
wherein the sensor device and flexible carrier are positioned on the target substrate.

19. The monitoring system of claim 18, wherein the target substrate is a heart.

20. The monitoring system of claim 19, wherein the sensor device provides real-time stiffness measurements with an accuracy of up to 96.7%.

21. The monitoring system of claim 18, wherein the sensor is at an implantation depth of at least 4 cm.

22. An implantable cardiac sensor comprising:
a biocompatible flexible carrier configured for implantation on a myocardial surface;
a harvesting module comprising a power harvesting element including one or more piezoelectric elements selected from the group consisting of poly(vinylidene fluoride) barium titanate (PVDF-BaTiO₃) fibers, poly(vinylidene fluoride) (PVDF) fibers, poly(vinylidene fluoride) (PVDF) films, polymethyl methacrylate (PMMA), lead zirconate titanate (PZT) films, and combinations thereof;
a sensor module having a sensor element, the sensor element comprising piezoelectric MEMS sensors configured to detect myocardial deformation; and
a controller module having circuitry operably connected to the harvester module and the sensor module, the controller module being configured to receive power from the power harvesting element, to receive sensor signals from the sensor element, and to wirelessly communicate an output signal corresponding to the sensor signal, the controller module being powered continuously with power harvested from the power harvesting element to generate and communicate the output signal;
wherein the circuitry includes maximum power point tracking (MPPT) control circuitry and power conversion circuitry configured to regulate harvested electrical energy for continuous battery-free system operation at ultra-low power levels and derive one or more of ejection fraction, cardiac stiffness and cardiac output parameters to communicate as data in the output signal.
